(19) United States
(12) Patent Application Publication
(10) Pub. No.: US 2006/0158337 A1
(43) Pub. Date: Jul. 20, 2006

(54) Remotely controlled weight scales

(76) Inventors: Martin A. Cohen, Ridgefield, CT (US); Vito James Carlucci, Stratford, CT (US)

Correspondence Address: STEVEN A. GARNER, ESQ.;CONAIR CORPORATION, ONE CUMMINGS POINT ROAD, STAMFORD, CT, 06902, US

(21) Appl. No.: 11/305,400

(22) Filed: Dec. 16, 2005

Related U.S. Application Data
(60) Provisional application No. 60/636,774, filed on Dec. 16, 2004.

(51) Int. Cl. G08B 21/00; A61B 5/05; G01G 23/00

(52) U.S. Cl. 340/666; 600/372; 600/547; 702/173; 700/305; 177/245

(57) ABSTRACT

A weight scale (5) has a cordless remote control (1) that utilizes an IR remote connection and an LED screen (6) in the handheld control (1) that displays data related to the function of the scale (5) and which features an array of control buttons (2) each dedicated to measured or calculated parameters.

### CROSS-REFERENCE TO RELATED APPLICATIONS

This application claims priority from U.S. Provisional patent application 60/636,774, filed on Dec. 16, 2004.

### BACKGROUND OF THE INVENTION

1. Field of the Invention

The present invention relates to weight scales and, more particularly, to remote control input and output devices associated with weight scales.

2. Description of Related Art

It is known in the art to provide devices that weigh the body and also to combine such weighing instruments with sensors that measure additional parameters. Other direct measurements, such as the impedance, from foot to foot, have been made on prior art scales and reported on the scale interface. In addition, prompted and manually input information, such as height, sex and age, has been processed by on-board information processors with directly measured parameters to calculate an extended array of relevant and useful numerical conclusions. Prior art weighing machines equipped with microprocessors have calculated parameters such as, body mass index, weight/height ratios, muscle mass, bone density, and fitness levels. Such “smart” scales aid exercise programs by comparing currant weight to personal goals, memorizing and comparing past measurements against current measurements, calculating the change and rate of change of monitored parameters. The viewing interfaces on these prior art, “smart” scales are, however, substantially complicated. The viewing windows are generally crowded with menus and data and are, therefore, difficult to read. Furthermore, the common size and shape of typical household scales make the viewing of the visual interface cramped and difficult. There is therefore a need for a user interface that can be comfortably handheld while standing upon a floor scale. Prior art solutions have attempted to solve this problem with cable connections, however, such a construction is limited by the reach of the cable and it requires an unsightly cable to stow away.

### OBJECTS OF THE INVENTION

It is desirable, therefore, to provide a convenient, cordless interface between a “smart” scale and a handheld controller. It is also desirable to design an understandable and intuitive operating logic that allows information to be easily read and managed. These and other objectives are achieved by the invention described herein.

### BRIEF SUMMARY OF THE INVENTION

The present invention is directed to a novel arrangement of a weight scale with a cordless remote control. The cordless controller In the preferred embodiment utilizes an IR remote connection and an LED screen in the handheld controller that is dedicated to a single parameter at one time and which features an array of control buttons each dedicated to a single measured or calculated parameter.

### BRIEF DESCRIPTION OF THE DRAWINGS

FIG. 1 is a schematic illustration of a preferred embodiment of the assembled handheld remote controller and remote scale interface according to the present invention.

FIG. 2 is a schematic illustration of a scale with remote communication and metallic pads for reading body impedance according to a preferred embodiment of the present invention.

FIG. 3 is a schemataic illustration of “smart” scale and handheld controller in line of sight communication according to a preferred embodiment of the present invention.

### DETAILED DESCRIPTION OF THE PREFERRED EMBODIMENTS OF THE INVENTION

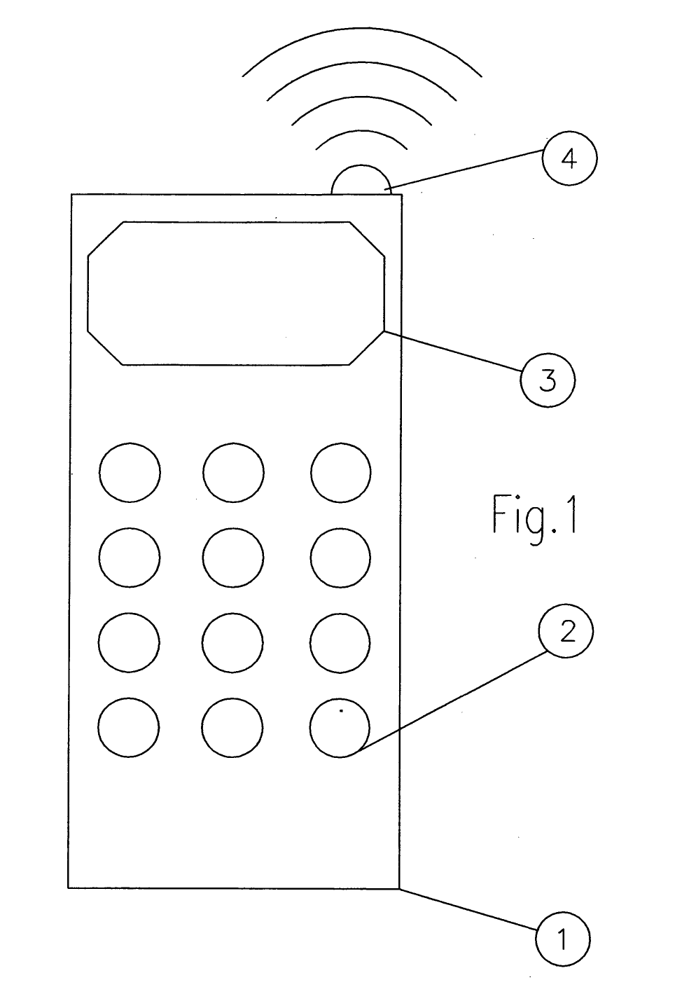

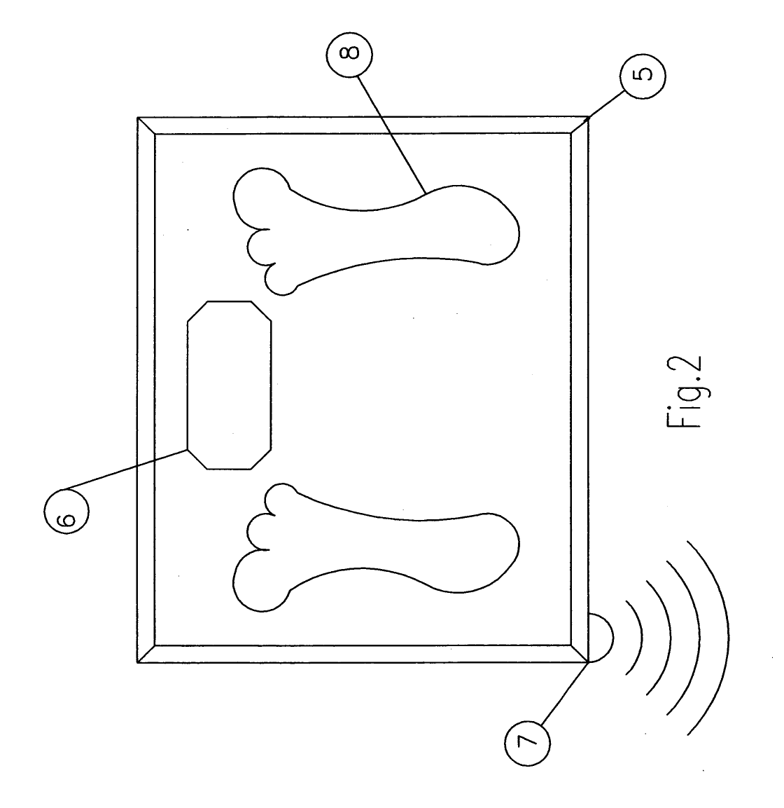

Referring to FIG. 1 and FIG. 2, an improved weight scale with a handheld remote controller according to a preferred embodiment of the present invention is illustrated. The handheld remote (1) is shown in FIG. 1. In the preferred embodiment the remote controller (1) will have a visual screen (3) with either an LED or LCD display. In the preferred embodiment the display will be dedicated to a single numerical parameter, for example, weight, body impedance, percent body fat, or fitness level. The menu for the various measurements is called out on the array of button switches (2) on the handheld controller (1). This is preferred to scrolling through a visual menu on the visual display. Scrolling menus are an alternative to the buttons, however, and may be combined with dedicated buttons to allow input of non-measured parameters such as: age, height and sex. The remote controller also features a communications port, which, in the preferred embodiment, would consist of an infrared transmitter and receiver (4), of conventional components, housed in the controller (1).

FIG. 2 depicts a top view of a weight scale (5). The scale features a view port (6) with an electronic display and a remote transmitter or receiver (7). The illustration also depicts a pair of metal contacts (8) which to make contact to each foot and then through the body in order to read impedance and thus be able to calculate percent body fat.

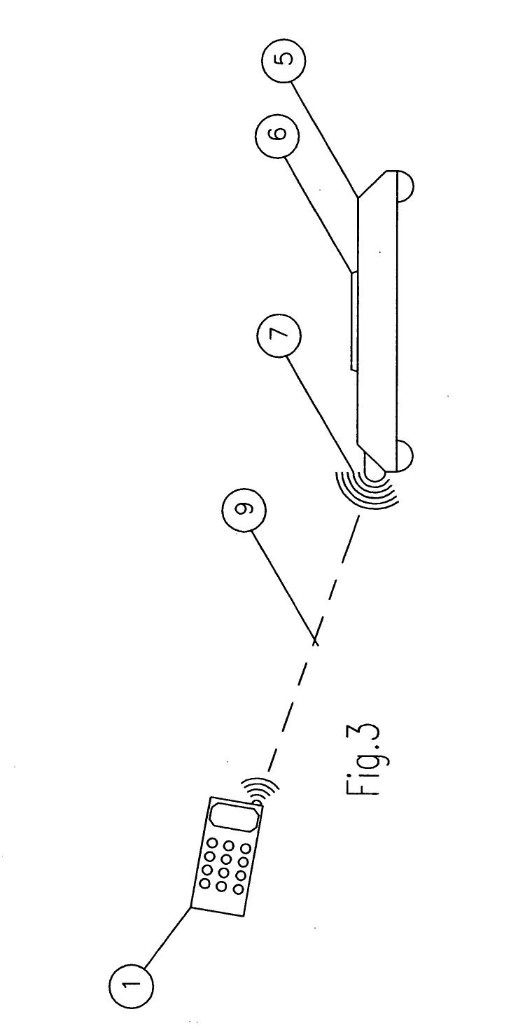

The two components, the remote controller (1) and the “smart” scale (5) which are linked together in the preferred embodiment with IR senders and receivers (4) & (7) would simply have to be within line of sight (9) to call up and calculate a range of information as depicted in FIG. 3.

While a preferred embodiment of the invention has been herein disclosed and described, it is understood that various modifications can be made without departing from the scope of the invention.

What is claimed is:

1) A weight scale system comprising
a scale unit adapted to receive an object to be weighed, said scale unit having an electronic control element and a data storage element; and
a remote unit adapted to communicate with said scale unit in order to send or receive data associated with the function of said scale unit.

2) A system according to claim 1, further comprising
a display screen on said remote unit adapted to display data related to the function of said scale unit.

3) A system according to claim 1, further comprising
user input controls on said remote unit adapted to receive user input associated with said scale unit and to transmit said input to said scale unit.

4) A system according to claim 1, further comprising
at least one wireless transmission element and at least one wireless receiver element for, respectively, transmitting and receiving data between of said scale unit and said remote unit.

5) A system according to claim 1, further comprising
a first wireless transmission element and a first wireless receiver element, each associated with said scale unit; and
a second wireless transmission element and a second wireless receiver element, each associated with said remote unit, such that said scale unit and said remote unit are adapted to send and receive data to and from each other.

6) A weight scale system comprising
a scale unit adapted to receive an object to be weighed, said scale unit having an electronic control element, a data storage element, and a display screen; and
a remote unit adapted to communicate with said scale unit;
a first transmission element associated with said scale unit and adapted to transmit data to said remote unit;
a first receiver element associated with said scale unit and adapted to receive data from said remote unit;
a second transmission element associated with said remote unit and adapted to transmit data to said scale unit; and
a second receiver element associated with said remote unit and adapted to transmit data to said scale unit.

7) A system according to claim 6, wherein
said display screen is adapted to display data relating to a function of said scale unit.

8) A system according to claim 6, wherein
said remote unit and said scale unit communicate wirelessly.

9) A system according to claim 6, further comprising
user input controls on said remote unit adapted to receive user input associated with said scale unit and to transmit said input to said scale unit.

10) A system according to claim 6, further comprising
electrically-conductive contact surfaces on said scale unit for enabling electric current to pass through a body that simultaneously contacts said contact surfaces in order to enable said scale unit to display, or compute and transmit to said remote unit for display thereon, data related to said body.
